(19) United States
(12) Patent Application Publication
(10) Pub. No.: US 2018/0308582 A1
(43) Pub. Date: Oct. 25, 2018

(54) DEVICE, SERVER, AND METHOD FOR ACQUISITION AND PROCESSING OF ALLERGY INFORMATION

(71) Applicant: BOE TECHNOLOGY GROUP CO., LTD., Beijing (CN)

(72) Inventors: Jiantao LIU, Beijing (CN); Wenchu DONG, Beijing (CN)

(21) Appl. No.: 15/852,277

(22) Filed: Dec. 22, 2017

(30) Foreign Application Priority Data
Apr. 20, 2017 (CN) 201710260679.1

(51) Int. Cl. G16H 50/20; G16H 10/60; G16H 70/60; H04W 4/029

(52) U.S. Cl. CPC G16H 50/20; H04W 4/029; G16H 70/60; G16H 10/60

(57) ABSTRACT

The application relates to a device, a server and a method for acquiring and processing allergy information. The device comprises: positioner for determining position information of the device; allergen detector for detecting allergen information in the environment where the device is located; physical sign detector for detecting physical sign information of a user of the device; memory for storing the position information, the allergen information and the physical sign information; communicator for sending out information; and controller configured to determine whether the physical sign information coincides with the abnormality determination condition, and send out the position information, the allergen information, and the abnormality information by means of a communicator when it is determined that the physical sign information coincides with the abnormality determination condition.

### CROSS-REFERENCE TO RELATED APPLICATION

This application claims priority to Chinese Patent Application No. 201710260679.1, filed on Apr. 20, 2017, the entire contents of which are incorporated herein by reference.

### TECHNICAL FIELD

Embodiments of the present disclosure relate to devices, servers and methods for the acquisition and processing of allergy information.

### BACKGROUND

Inhalation allergens such as pollen, catkins, dust, insects and mites, and animal dander may cause respiratory diseases such as asthma, cough, rhinitis, bronchitis, and the like. A direct way to prevent and treat respiratory allergic diseases is to purify the air and remove allergens in the air inhaled into a human body.

### SUMMARY

Embodiments of the present disclosure provide a device, a server, and a method for the acquisition and processing of allergy information.

According to a first aspect of the present disclosure, there is provided a device for acquiring allergy information, comprising: a positioner for determining position information of the device; an allergen detector for detecting allergen information in an environment where the device is located; a physical sign detector for detecting physical sign information of a user of the device; a memory for storing the position information, the allergen information and the physical sign information; a communicator for sending out information; and a controller configured to determine whether the physical sign information meets an abnormality determination condition, and when it is determined that the physical sign information meets the abnormality determination condition, to send out the position information, the allergen information and abnormality information for determining that the physical sign information meets the abnormality determination condition by means of the communicator.

According to a second aspect of the present disclosure, there is provided a server for processing allergy information, comprising: a communicator for performing communication; a memory for storing information received by the communicator; and a controller configured to receive, from at least one of the devices according to any of the preceding aspects, the position information, allergen information and abnormality information through the communicator and forward the received position information, allergen information, and abnormality information to the other corresponding devices through the communicator.

According to a third aspect of the present disclosure, there is provided a method for processing allergy information, comprising: receiving, by a controller of a server for processing allergy information, the position information, allergen information and abnormality information from at least one of the devices according to the aforementioned first aspect through a communicator, and forwarding the received position information, allergen information and abnormality information to the other corresponding devices through the communicator.

### BRIEF DESCRIPTION OF THE DRAWINGS

In order to illustrate the technical solution of the embodiments of the present disclosure more clearly, the drawings of the embodiments will be briefly described as follows. The drawings described below relate only to some embodiments of the disclosure and are not intended to be limitation of the disclosure.

FIG. 1 is a schematic block diagram of a device according to an embodiment of the present disclosure.

FIG. 2 is a flow chart of a method performed by the device according to an embodiment of the present disclosure.

FIG. 3 is a schematic block diagram of a server according to an embodiment of the present disclosure.

FIG. 4 is a flow diagram of a method performed by a server according to an embodiment of the present disclosure.

FIG. 5 is another flow diagram of a method performed by a server according to an embodiment of the present disclosure.

### DETAILED DESCRIPTION

In order to make the object, technical solution and advantages of the embodiments of the present disclosure clearer, the technical solution of the embodiments of the present disclosure will be described below in a clear and comprehensive manner with reference to the accompanying drawings of the embodiments of the present disclosure. Obviously, the described embodiments are only a part, rather than all, of the embodiments of the present disclosure. All the other embodiments obtained by an ordinary person skilled in the art based on the embodiments described in the present disclosure without needing to pay any creative effort are within the scope of protection of the present disclosure.

As mentioned above, embodiments of the present disclosure provide a device, a server, a system and a method for acquisition and processing of allergy information, which can provide a flexible solution directed to allergens. In the following parts, the device, the server, the system and the method of the present disclosure will be described in detail with reference to the corresponding embodiments.

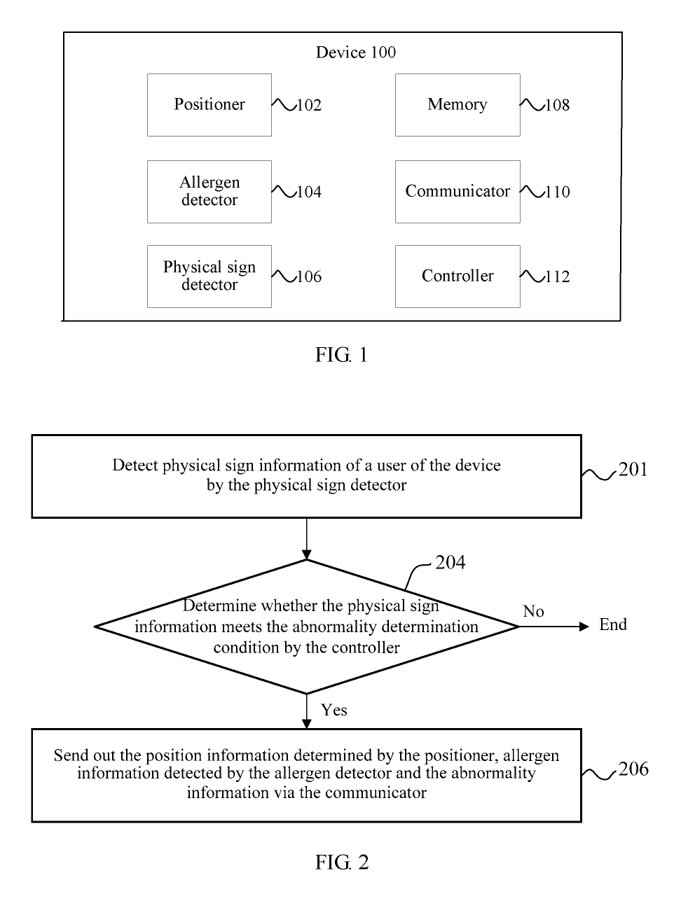

FIG. 1 is a schematic block diagram of a device according to an embodiment of the present disclosure. As illustrated by FIG. 1, a device 100 for acquiring allergy information includes a positioner 102, an allergen detector 104, a physical sign detector 106, a memory 108, a communicator 110, and a controller 112.

The positioner 102 is capable of determining the position information of the device 100. As an example, the positioner 102 may be a GPS positioning device or a Beidou positioning device. As another example, the positioner 102 may also be implemented by using an LBS (location-based service) technology of a mobile communication system. However, the present disclosure will not make particular limitations in this respect. The positioner 102 may be implemented using a variety of related or developed mobile positioning technologies.

The allergen detector 104 is capable of detecting the allergen information in an environment where the device 100 is located. The term “allergen” used in this disclosure may encompass various sources that may cause respiratory system diseases (also referred to herein as “respiratory diseases”). For example, an allergen may be a source that causes people with allergic constitution to have symptoms of allergic reactions (e.g., asthma, cough, rhinitis, bronchitis, etc.), examples of which include, but are not limited to, pollen, catkins, dust, insects and mites, animal dander, etc. Also, for example, allergens may also be sources that cause people to develop respiratory diseases (such as cold) spread through air, examples of which include, but are not limited to, fungi, viruses, and the like. Accordingly, the term “allergen information” herein is information related to the presence or absence of allergens. For example, the allergen information may include the name of the allergen and the presence or absence of the allergen. For another example, the allergen information may include a name of the allergen and an amount of the allergen in the air.

As an example, the allergen source detector 104 may include an related sensor for detecting dust. For example, an air quality sensor developed by Mitsubishi Electric that utilizes a laser to detect PM 2.5 concentration can be used to identify pollen and dust. As another example, the allergen source detector 104 may include an related sensor for detecting a virus. For example, a nanowire field-effect transistor sensor for detecting cold viruses as developed by Fernando Patolsky et al. can be utilized (see Fernando Patolsky et al., “Electrical detection of single viruses”, PNAS, Vol. 101, No. 39, 2004). However, the present disclosure shall not make particular limitations in this respect. The allergen detector 104 may be implemented using a variety of related or developed technologies for detecting an allergen.

The physical sign detector 106 is capable of detecting the physical sign information of the user of the device 100. Examples of physical sign information include, but are not limited to, pulse, blood pressure, respiratory rate, body temperature, electrocardiogram, and the like. For example, the physical sign detector 106 may include a wearable vital sign sensor module, Silmee, developed by Toshiba that can detect the wearer's pulse, electrocardiogram and body temperature. Also, for example, the physical sign detector 106 may include a polyvinylidene fluoride (PVDF) type piezoelectric thin film sensor capable of detecting blood pressure and respiratory rate (see Xin Yi et al., “Wearable Health Monitoring Body Area Network System based on Piezoelectric Film Sensors”, Journal of National University of Defense Technology, Vol. 38, No. 6, 2016). However, the present disclosure will not make particular limitations in this respect. The physical sign detector 106 may be implemented using a variety of related or developed physical sign detection sensors.

The memory 108 is capable of storing information. For example, the memory 108 may store the position information determined by the positioner 102, the allergen information detected by the allergen detector 104, and the physical sign information detected by the physical sign detector 106. The memory 108 may also store a program executed by the controller 112 to implement the corresponding function of the device 100, as will be described later. The memory 108 may be implemented using any related or developed storage device.

The communicator 110 is capable of sending out information. For example, the communicator 110 may be a commercially available wireless communication module for a mobile phone. However, the present disclosure will not make particular limitations in this respect. The communicator 110 may be implemented using any related or developed wireless communication technology.

The controller 112 is configured to determine whether or not the physical sign information meets the abnormality determination condition, and when it is determined that the physical sign information meets the abnormality determination condition, send out the position information, the allergen information and the abnormality information for determining that the physical sign information meets the abnormality determination condition through the communicator 110. The controller 112 may be implemented as at least one processor configured to implement the aforementioned function by executing a program stored in the memory 108. FIG. 2 is a flow chart of the method performed by the device 100, which contains the steps of the program executed by the controller 112. In the following part, the implementation details of the controller 112 will be described in detail with reference to the flow chart of FIG. 2.

As illustrated by FIG. 2, the method includes steps 202 and 204. At step 202, the physical sign detector 106 of the device 100 detects the physical sign information of the user of the device 100. The physical sign detector 106 and its detected physical sign information have been described in detail above and will not be described further.

In step 204, it is determined by the controller 112 of the device 100 whether or not the physical sign information meets the abnormality determination condition. As an example, the abnormality determination condition may be: the current physical sign information of the user is within a value range outside the normal range of the physical sign information. That is, it is possible to simply compare the current physical sign information of the user detected by the physical sign detector 106 with the normal range of the physical sign information to determine whether or not the user is in an abnormal state. As another example, the abnormality determination condition may be: the current sign information of the user matches with the pattern of the physical sign information of a certain disease. For example, for each of a variety of diseases (e.g., asthma, cough, rhinitis, bronchitis, colds, etc.) that may be caused by allergens, data of physical sign information (pulse, blood pressure, respiratory rate, body temperature, electrocardiogram, etc.) of a certain number of patients can be obtained from a medical institution as the sample data. Data mining techniques can be used to dig out the patterns specific to each disease (for example, the specific value range of the respiratory rate, the specific electrocardiogram shape, etc.) from the sample data of these diseases. The correspondence table for each disease and its corresponding pattern obtained accordingly can be stored in the memory. The controller 112 may determine whether or not the user is in an abnormal condition by comparing the current physical sign information of the user detected by the physical sign detector 106 with the respective patterns in the correspondence table. As yet another example, the above two examples may be combined. That is, in the case where it is impossible to determine whether or not the user is in an abnormal condition by the correspondence table, it is also possible to determine whether or not the user is in an abnormal condition by comparing the current physical sign information of the user detected by the physical sign detector 106 with the normal range of the physical sign information in an abnormal condition.

If it is determined in step 204 that the physical sign information does not meet the abnormality determination condition, the method may end. On the other hand, if it is determined in step 204 that the physical sign information meets the abnormality determination condition, the controller 112 will send out the position information determined by the positioner 102 of the device 100, the allergen information detected by the allergen detector 104, the abnormality information for determining that the physical sign information meets the abnormality determination condition through the communicator 110 in step 206. As an example, the position information, the allergen information, and the abnormality information may be sent to the server for processing allergy information such that the position information, the allergen information, and the abnormality information can be forwarded to other corresponding devices 100.

In the above example of comparing with the normal range of the physical sign information, the abnormality information may include, for example, information that “the user is already in an abnormal condition” without including the name of the disease. Alternatively, the allergen source information detected by the allergen detector 104 may be further considered to determine the name of the disease. For example, if allergen information indicates that there is pollen in the environment, the name of disease corresponding to pollen allergy can be determined. For another example, if the allergen information indicates that there is a cold virus in the environment, the name of the disease can be identified as a cold.

In the above example of making a comparison between the respective patterns in the correspondence table, the abnormality information may include the determined name of disease. Similarly to the above, the determined name of disease may be further combined with the allergen information detected by the allergen detector 104 to further improve the accuracy of the abnormality determination step.

As for the positioner 102, it may determine the position information of the device 100 in real time, or determine the position information of the device 100 after determining that the user is in an abnormal condition. As for the allergen detector 104, it can detect the allergen information in the environment in real time. In this way, users can know the situation in the environment in real time. Alternatively, the allergen detector 104 may also detect the allergen information in the environment after determining that the user is in an abnormal condition. The details of the server for processing allergy information will be described later.

In this way, since the position information, the allergen information and the abnormality information detected by the device 100 can be forwarded to the other corresponding devices 100, the user of the other devices 100 can know in real time a position where the allergen exists currently and which has caused an abnormal condition so that a preventive measure can be prepared in advance or activities in the position of the allergen can be avoided.

As another example, the device 100 may be presented as an allergen detector 104, a physical sign detector 106, and an App application on a cell phone, as will be described later. In this case, the device 100 may also transmit the position information, the allergen information, and the abnormality information to the relatives and friends equipped with the device 100 by a cell phone, for example, in the form of a short message. In other words, “sending out” mentioned in step 206 is not limited to forwarding to other devices 100 through the server.

Alternatively, the controller 112 may be configured to send the position information and the allergen information of the device 100 to the server through a communicator 110 (e.g., by executing a program executed by a memory 108) when it is determined that the physical sign information does not meet the abnormality determination condition or in response to a request from the server. This allows the server to be able to know the allergy situation at the position of each device 100 in real time, or when needed. Moreover, in the former case, when the user is not in an abnormal condition immediately because of the existence of the allergen, the server is able to make a pre-warning to other corresponding devices 100 based on the allergen information obtained in real time.

Optionally, the controller 112 may be configured to send a historical record of the position information, allergen information and abnormality information stored in the memory 108 to the server through the communicator 110 (e.g., by executing a program in the memory 108) in response to a request from the server. A historical record may include the position information, allergenic information, and abnormality information corresponding to an abnormal condition detection result. In this way, the server is able to predict in which positions an abnormal condition might occur currently according to a historical record of abnormal condition of a user of each device, thereby making a pre-warning to each user.

Optionally, the controller 112 may be configured to receive prompt information from the server via the communicator 110 (e.g., by executing a program in the memory 108), the prompt information including information of a position where an abnormal condition has occurred and the corresponding allergen information. As will be described later on the server, the prompt information may be the reporting information received from other devices 100 and forwarded by the server or may be information predicted by the server based on the historical records of the position information, the allergen information and the abnormality information of multiple devices 100. The prompt information may take various forms including, but not limited to, visual information (e.g., text, image), auditory information (e.g., voice, ringing sound), tactile information (e.g., vibration), and the like. This allows a user of a device 100 to know in real time a position where the allergen exists currently and which has caused an abnormal condition so that a preventive measure can be prepared in advance or activities in the position of the allergen can be avoided.

Optionally, the controller 112 may be configured to obtain medical data of respiratory diseases from a medical institution or a disease prevention center via the communicator 110 (e.g., by executing a program in the memory 108), the medical data of respiratory diseases including allergen information and the corresponding information of respiratory disease. For the current respiratory disease caused by an allergen (such as influenza), the medical institution will have a record of patient's visits, and/or the disease prevention center will have pre-warning information. The record of patient's visits or the pre-warning information may correspond to the medical data of the respiratory disease. The controller 112 may actively query or passively receive the respiratory the medical data of the respiratory disease from the medical institution or the disease prevention center through the communicator 110. This allows the user of the device 100 to know what abnormal conditions exist locally at present, so that the preventive measures can be prepared in advance.

Each of the functional parts of the device 100 has been described above. As for the shape of the device 100, for example, the main body of the device 100 may be similar to the shape of a wristwatch. The positioner 102, the memory 108, the communicator 110, and the controller 112 may be included in the main body. The allergen detector 104 may be attached to the use's clothes and communicate wirelessly with the subject (e.g., via Bluetooth), or may also be integrated on the main body. The physical sign detector 106 may be attached to a user's body (e.g., adhered to a user's chest) and communicate wirelessly with the main body (e.g., via Bluetooth), or may also be integrated on the main body (e.g., attached to the wrist).

As another example, the device 100 may be presented as an allergen detector 104, a physical sign detector 106, and an App application of a cell phone. The allergen detector 104 may be attached to the user's clothes and communicate wirelessly (e.g., via Bluetooth) with the App application of the cell phone. The physical sign detector 106 may be attached to the user's body (e.g., adhered to the user's chest) and communicate wirelessly with the App application of the cell phone. The App application of the cell phone can obtain position information from a positioning module in the cell phone and communicate with the server through a communication module in the cell phone. This may make full use of the popularity of the cell phone and is helpful for promoting the device 100. It should be noted that the cell phone is just an example, and other mobile terminals can also be adopted.

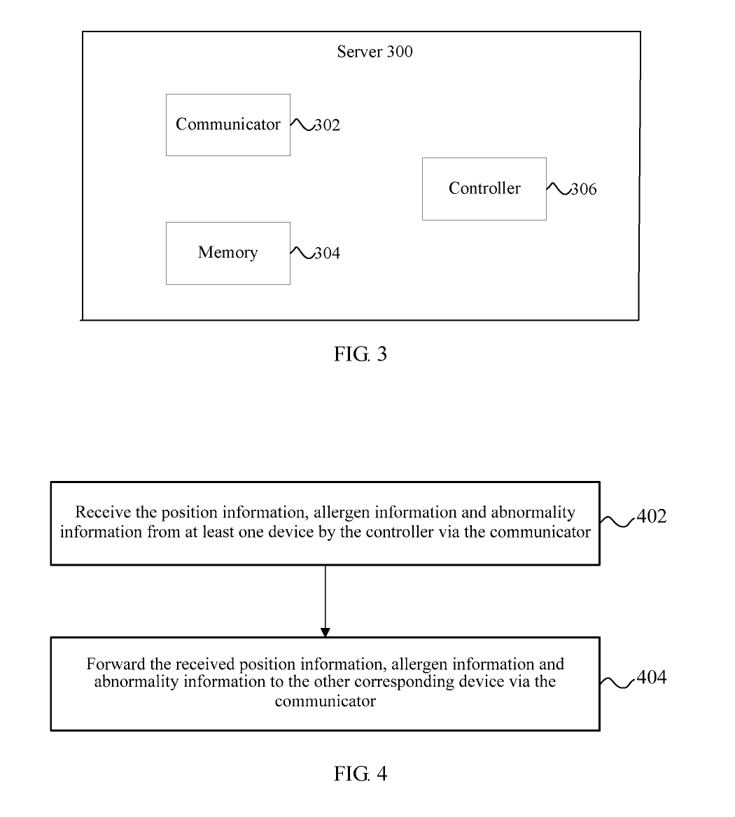

FIG. 3 is a schematic block diagram of a server according to an embodiment of the present disclosure. As illustrated by FIG. 3, the server 300 for processing the allergy information includes a communicator 302, a memory 304, and a controller 306.

The communicator 302 is capable of performing communication. For example, the communicator 302 may adopt a commercially available communication module for a cloud server. However, the present disclosure will not make a particular limitation in this respect. The communicator 302 may be implemented as various related or developed communication modules for the server.

The memory 304 is capable of storing information. For example, the memory 304 may store the reporting information obtained from various devices 100. The memory 304 may also store a program executed by the controller 306 to implement the corresponding function of the server 300, as will be described later. The memory 304 may be implemented as any related or developed storage device.

The controller 306 is configured to receive the position information, the allergen information and the abnormality information from at least one of the devices 100 through the communicator 302 and forward the received position information, allergen information and abnormality information to other corresponding devices 100 through the communicator 302. The controller 306 may be implemented as at least one processor configured to implement the functions by executing a program stored in the memory 304. The steps of the program executed by the controller 306 are illustrated by FIG. 4. The details of the implementation of the controller are described in detail with reference to the flow chart of FIG. 4.

In step 402, the controller 306 receives the position information, the allergen information and the abnormality information from the at least one of the devices 100 through the communicator 302. This step can be implemented based on the server-client architecture, which will not be described in detail.

In step 404, the received location information, allergen information, and abnormality information are forwarded by the controller 306 to other corresponding devices 100 via the communicator 302. Similar to step 402, this step may also be implemented based on a server-client architecture. For example, the other devices 100 may be in the same geographical range (e.g., city, township, town, etc.) as the device 100 reporting the information. For another example, the corresponding other device 100 may be a device 100 that intends to travel to the geographic range where the device 100 reporting the information is located and which has sent a request for query to the server 300 accordingly.

Thus, according to the method illustrated by FIG. 3, the user of the other device 100 can know in real time the position where the allergen exists currently and which has caused an abnormal condition so that a preventive measure can be prepared in advance or activities in the position of the allergen can be avoided.

Optionally, the controller 306 may be configured to receive the position information and the allergen information from the respective device 100 in real time or as needed, for example, by executing a program in the memory 304. In this way, the server can know the situation of the allergen of each device 100 in real time, or when needed. Moreover, in the former case, even if the user of the device 100 reporting the information is not in an abnormal condition, the server can still make a pre-warning to the corresponding other device 100 based on the allergen information obtained in real time. In the latter case, the server is able to make a pre-warning to the corresponding device 100 by comparing the allergen information received from the device 100 with the historical record of the abnormal condition of the local and/or remote area, as will be described later.

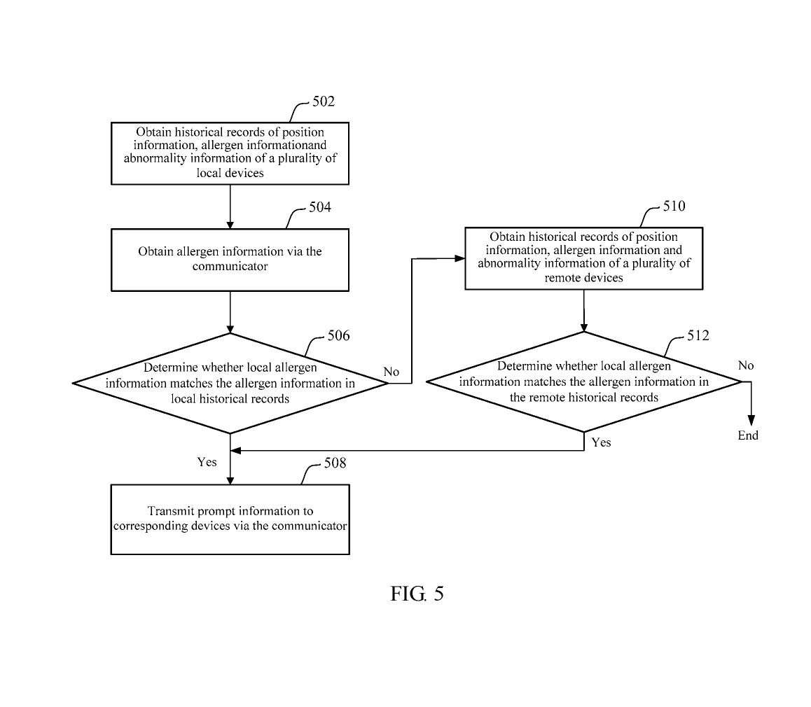

Optionally, the controller 306 may be configured to execute the corresponding functions by executing at least some of the steps of the program stored in the memory 304 as illustrated by FIG. 5. The implementation details will be described in detail according to the flow chart of FIG. 5.

As an example, the controller 306 executes steps 502, 504, 506, and 508 of FIG. 5. In step 502, the controller 306 acquires the historical records of the position information, the allergen information and the abnormality information of the plurality of local devices 100. As mentioned earlier, a historical record (also referred to in this text as “history of an abnormal condition” or “historical record of an abnormal condition”) may include position information, allergen information and abnormality information corresponding to an abnormal condition detection result.

For example, the controller 306 may accumulate the position information, the allergen information and the abnormality information previously reported by the respective device 100 in the memory 304. For example, the historical record of these abnormal conditions can be stored in the form of a database. In this way, the controller 306 may obtain a historic record of the abnormal conditions of a plurality of local devices 100 from the database. The above-mentioned “a plurality of local devices 100” refers to a plurality of devices 100 in the same geographical range (e.g., city, township, town, etc.) as a device 100 to which a pre-warning is to be send. For another example, the controller 306 may also acquire the stored historic records of abnormal conditions from the plurality of local devices 100 by the communicator 302 when necessary.

At step 504, the controller 306 acquires local allergen information via the communicator 302. For example, local allergen information may be obtained from at least one of the following sources: a disease prevention center (e.g., a disease management and pre-warning center), an environment monitoring center, and a plurality of local devices 100.

At step 506, the controller 306 determines whether the local allergen information matches the allergen information in the local historical record. For example, the local allergen information obtained from at least one of the above sources may be compared with the allergen information in the local historical record. In the case where the local allergen information obtained by two or three of the above sources is compared with the allergen information in the local historical record, it can be deemed that matching exists as long as any one of the two or three sources causes matching. The above-mentioned “matching” may refer to the sameness of name of the allergen, or may refer to that the name of allergen is the same and the difference of content is within a predetermined range of threshold, or may refer to that the name of allergen is the same and the current content is greater than or equal to the content in the historic record.

If it is determined in step 506 that the local allergen information and the allergen information in the local historical record do not match, the method of the example may end. On the other hand, if it is determined in step 506 that the local allergen information matches the allergen information in the local historical record, the controller 306 sends prompt information to corresponding devices 100 via the communicator 302 in step 508. For example, the corresponding device 100 may be local devices 100. For another example, the corresponding devices 100 may be devices 100 intending to travel to the local devices 100 and having sent a request for query to the server 300 accordingly. As mentioned earlier, the prompt information may take various forms including, but not limited to, visual information (e.g., text, image), auditory information (e.g., voice, ringing sound), tactile information (e.g., vibration), and the like. In this way, the server can predict in which positions an abnormal condition might occur according to the history of abnormal condition of the user of each local device 100, so as to make a pre-warning to each user.

As another example, the controller 306 also performs steps 510 and 512 in addition to steps 502, 504, 506, and 508 in FIG. 5. That is, if it is determined in step 506 that the local allergen information does not match the allergen information in the local historical record, the controller 306 will acquire the position information, the allergen information and the historical record of the abnormality information of a plurality of remote devices 100. “A plurality of remote devices 100” refer to a plurality of devices 100 in a geographical range (e.g., city, township, town, etc.) different from the geographical range of a device 100 to which a pre-warning is to be provided. Similar to the step 502, the controller 306 may acquire a historical record of the abnormal conditions of the plurality of remote devices 100 from the database in the memory 304 or acquire the stored historical record of abnormal conditions from the plurality of remote devices 100 via the communicator 302.

At step 512, the controller 306 determines whether the local allergen information matches the allergen information in the remote historical record. Step 512 is similar to step 506 and will not be described in detail here. If it is determined in step 512 that the local allergen information does not match the allergen information in the remote historical record, the method of the example may end.

On the other hand, if it is determined in step 512 that the local allergen information matches the allergen information in the remote historical record, the controller 306 will send prompt information to the corresponding device 100 via the communicator 302 in step 508. The Step 508 has been described in detail above and will not be repeated here. In this way, the server may predict in which positions the abnormal condition might occur currently by taking into account a historical record of the abnormal condition of each remote device 100, thereby giving a more comprehensive pre-warning to each user.

As another example, in addition to the steps in FIG. 5, the controller 306 also performs steps 510 and 512 in the case where it is determined in step 506 that a match exists. In this way, the server can make a more comprehensive pre-warning.

The method according to the embodiments of the present disclosure can be performed periodically or in response to the request from the server or the user.

According to the above description, at least one embodiment of the present disclosure also provides a system for acquiring and processing allergen information comprising a plurality of aforementioned devices 100; and the server 300 described above.

At least one embodiments of the present disclosure further disclose a method for obtaining allergy information, comprising: detecting, by a physical detector of a device for acquiring allergy information, physical sign information of the user of the device; determining, by a controller of the device, whether the physical sign information meets an abnormality determination condition, and when it is determined that the physical sign information meets the abnormality determination condition, sending out position information determined by a positioner of the device, allergen information detected by an allergen detector of the device and abnormality information for determining that the physical sign information meets the abnormality determination condition by a communicator of the device.

In at least one embodiments, the position information, the allergen information and the abnormality information can be transmitted through the communicator of the device to a server for processing allergy information such that the position information, the allergen information and the abnormality information can be forwarded to other corresponding devices for obtaining allergy information.

In at least one embodiments, the position information and the allergen information can be transmitted by the controller to the server by means of the communicator in a case where it is determined that the physical sign information does not meet the abnormality determination condition or in response to a request from the server.

In at least one embodiments, a historical record of the position information, the allergen information and the abnormality information stored in the memory can be transmitted by the controller to the server through the communicator in response to the request from the server.

In at least one embodiment, prompt information can be received by the controller from said server through said communicator, said prompt information including position information where an abnormal condition has occurred locally and corresponding allergen information.

In at least one embodiment, medical data of respiratory diseases can be obtained by the controller from a medical institution or a disease prevention center through said communicator, said medical data of respiratory diseases including allergen information and corresponding respiratory disease information.

It should be noted that at least some aspects of the present disclosure may be implemented as a system, method, or computer program product. Thus, some aspects of the present disclosure may be implemented in the following forms: complete hardware implementations (including application specific integrated circuits (ASICs), field programmable gate arrays (FPGAs), etc.), complete software implementations (including firmware, presence software, Microcode, etc.), or a combination of hardware and software. In addition, in some embodiments, various aspects of the present disclosure may also be implemented as one or more computer-readable media that contain computer-readable program code.

The foregoing content is merely illustrative of the present disclosure and is not intended to limit the scope of protection of the present disclosure. The scope of protection of the present disclosure is defined by the appended claims.

What is claimed is:

1. A device for acquiring allergy information, comprising:
a positioner configured to determine position information of the device;
an allergen detector configured to detect allergen information in an environment where the device is located;
a physical sign detector configured to detect physical sign information of a user of the device;
a memory configured to store the position information, the allergen information and the physical sign information;
a communicator configured to send out information; and
a controller configured to determine whether the physical sign information meets an abnormality determination condition, and when it is determined that the physical sign information meets the abnormality determination condition, to send out the position information, the allergen information and abnormality information for determining that the physical sign information meets the abnormality determination condition by means of the communicator.

2. The device according to claim 1, wherein the position information, the allergen information and the abnormality information are transmitted through the communicator to a server for processing allergy information such that the position information, the allergen information and the abnormality information can be forwarded to other corresponding devices for obtaining allergy information.

3. The device according to claim 2, wherein the controller is further configured to transmit the position information and the allergen information to the server by means of the communicator in a case where it is determined that the physical sign information does not meet the abnormality determination condition or in response to a request from the server.

4. The device according to claim 2, wherein said controller is further configured to transmit a historical record of the position information, the allergen information and the abnormality information stored in the memory to the server through the communicator in response to the request from the server.

5. The device according to claim 2, wherein said controller is further configured to receive prompt information from said server through said communicator, said prompt information including position information where an abnormal condition has occurred locally and corresponding allergen information.

6. The device according to claim 1, wherein said controller is further configured to obtain medical data of respiratory diseases from a medical institution or a disease prevention center through said communicator, said medical data of respiratory diseases including allergen information and corresponding respiratory disease information.

7. A server for processing allergy information, comprising:
a communicator configured to perform communication;
a memory configured to store information received by the communicator; and
a controller configured to receive, from at least one of the device according to claim 1, the position information, allergen information and abnormality information through the communicator and forward the received position information, allergen information, and abnormality information to other corresponding devices through the communicator.

8. The server according to claim 7, wherein said controller is further configured to:
obtain local historical records of the position information, allergen information and abnormality information of a plurality of local devices;
obtain local allergen information through the communicator;
determine whether the local allergen information matches the allergen information in the local historical records; and
when the local allergen information matches the allergen information in the local historical records, transmit prompt information to one or more of the other corresponding devices through the communicator.

9. The server according to claim 8, wherein obtaining the local allergen information through the communicator comprises at least one of:
obtaining local allergen information from a disease prevention center;
obtaining local allergen information from an environment monitoring center; or
obtaining detected allergen information from the plurality of local devices.

10. The server according to claim 8, wherein said controller is further configured to:
obtain remote historical records of position information, allergen information and abnormality information of a plurality of remote devices when the local allergen information does not match the allergen information in the local historical records;
determine whether the local allergen information matches the allergen information in the remote historical records; and
when the local allergen information matches the allergen information in the remote historical records, transmit prompt information to the one or more of the other corresponding devices through the communicator.

11. A method for processing allergy information, comprising:
receiving, by a controller of a server for processing allergy information, the position information, allergen information and abnormality information from at least one of the device according to claim 1 through a communicator, and forwarding the received position information, allergen information and abnormality information to other corresponding devices through the communicator.

12. The method according to claim 11, wherein the method further comprises:
obtaining, by the controller, local historical records of the position information, allergen information and abnormality information of a plurality of local devices;
obtaining, by the controller, local allergen information through the communicator;
determining, by the controller, whether the local allergen information matches the allergen information in the local historical records; and
in a case where the local allergen information matches the allergen information in the local historical records, transmitting prompt information to one or more of the other corresponding devices by the controller through the communicator.

13. The method according to claim 12, wherein obtaining, by the controller, the local allergen information through the communicator comprises at least one of:
obtaining local allergen information from a disease prevention center;
obtaining local allergen information from an environment monitoring center; or
obtaining detected allergen information from the plurality of local devices.

14. The method according to claim 12, wherein the method further comprises:
obtaining remote historical records of position information, allergen information and abnormality information of a plurality of remote devices by the controller when the local allergen information does not match the allergen information in the local historical records;
determining whether the local allergen information matches the allergen information in the remote historical records by the controller; and
in a case where the local allergen information matches the allergen information in the remote historical records, transmitting prompt information to the one or more of the other corresponding devices by the controller through the communicator.
